(19) United States
(12) Patent Application Publication
(10) Pub. No.: US 2021/0313037 A1
(43) Pub. Date: Oct. 7, 2021

(54) EARLY EXERCISE DETECTION FOR DIABETES MANAGEMENT

(71) Applicant: INSULET CORPORATION, Acton, MA (US)

(72) Inventors: Jason O'CONNOR, Acton, MA (US); Ian MCLAUGHLIN, Groton, MA (US); Daniel ALLIS, Boxford, MA (US); Paul Frederick BENTE, IV, Westford, MA (US); Steven CARDINALI, Tewksbury, MA (US); Joon Bok LEE, Acton, MA (US)

(21) Appl. No.: 16/840,603

(22) Filed: Apr. 6, 2020

(51) Int. Cl. G16H 20/30; G16H 10/60; G16H 20/17; G16H 40/63; G16H 50/50; A61M 5/168; G16H 30/20

(52) U.S. Cl. CPC G16H 20/30; G16H 10/60; G16H 20/17; G16H 40/63; A61M 2230/63; A61M 5/168; G16H 30/20; A61M 2230/201; G16H 50/50

(57) ABSTRACT

A system, techniques, and computer-readable media includes examples that provide an indication of an early exercise detection are described. An example of an early exercise detection application executed by a processor may cause the processor to perform functions and be operable to obtain image data including metadata from a camera of a mobile device during, for example, an unlock procedure of a mobile device. The processor may determine whether the obtained image data includes location or timestamp information in metadata or has image data that may be recognized as exercise-related objects. Based on the determinations, the processor may output an indication of early exercise detection to an artificial pancreas application, which is operable to adjust an amount of insulin to be delivered to a user.

### TECHNICAL FIELD

The disclosed examples generally relate to medication delivery for diabetes management. More particularly, the disclosed examples relate to techniques, processes, devices or systems for managing operation of a wearable drug delivery device based on an early detection of exercise.

### BACKGROUND

Types of drug delivery devices may include settings in a diabetes management application that allow for temporary adjustments to regular automatic insulin delivery. The diabetes management application settings may include a setting that permits the suspension of delivery of insulin. These types of drug delivery devices, however, are not capable of determining when a user is exercising. Specific instances of exercise may be weightlifting, fitness class participation, jogging, biking, kayaking, hiking, brisk walking, playing a game, such as tennis or racquetball, and so on.

Some diabetes management applications may allow users to manually adjust insulin settings prior to exercise and after exercise, but the users must remember to adjust the settings prior to exercising and further remember to turn it off afterward. If the user is in a rush or wants to spontaneously participate in a game or other exercise, the constant turning ON and OFF can be aggravating, easily forgotten, and may limit the user's participation in exercise to the user's detriment.

Other diabetes management applications may be more advanced and may have default temporary adjustments that are limited in the variability of the settings and may not be optimal for all users. For example, a diabetic child may respond differently to exercise than a diabetic adult, or a physically-fit adult diabetic who exercises frequently and over several years may respond differently to exercise than an adult diabetic who is only beginning to exercise.

### SUMMARY

An example of non-transitory computer readable medium embodied with programming code executable by a processor is provided. In the example, the processor when executing the programming code is operable to perform functions, including functions to obtain image data including metadata from a camera coupled to the processor. The processor may determine whether the obtained image data includes location information or timestamp information in the metadata. Based on a determination that the metadata includes location information, the location information may be evaluated for a correspondence to known exercise locations. The timestamp information may be evaluated for a correspondence to an exercise diary based on a determination that the metadata includes timestamp information. A correspondence with either an exercise location in the known exercise locations or an exercise time in the exercise diary may be identified. In response to an identification of a correspondence, an indication of early exercise detection may be output to an artificial pancreas application.

In another example, a system including a mobile device and wearable drug delivery device is provided. The mobile device may include a processor, a transceiver, a camera, and a memory. Programming code, an early exercise detection application and an artificial pancreas application may be stored in the memory. The programming code, the early exercise detection application and the artificial pancreas application stored in the memory are executable by the processor, and when executing the early exercise detection application, the processor is operable to obtain image data from the camera. The image data may include metadata that may be obtained by the camera during an unlock procedure of the mobile device. The metadata may include location information or timestamp information. The processor may determine whether the obtained image data includes location information or timestamp information. Based on a determination that the metadata includes location information or a timestamp, the processor may evaluate the location information for a correspondence to known exercise locations or evaluate the timestamp with respect to an exercise diary. The processor may identify a correspondence of the location information with an exercise location in the known exercise locations, the timestamp information with an exercise time in the exercise diary or an exercise-related object recognized in the image data with exercise-related objects in the known exercise locations. The processor, in response to an identification of a correspondence, may output an indication of early exercise detection to the artificial pancreas application. The wearable drug delivery device may be operable to deliver insulin to a user, and may include a communication interface device, a reservoir, a pump mechanism, a memory and a controller. The communication interface device may be operable to receive and transmit signals. The reservoir may be operable to store insulin. the pump mechanism may be coupled to the reservoir and operable to expel the stored insulin from the reservoir in response to control signals. The memory may be operable to store instructions. The controller may be operable to execute the instructions and control the communication interface device and the pump mechanism by outputting control signals and be communicatively coupled via the communication interface device to the transceiver and the processor of the mobile device. The controller, when executing the instructions, is operable to receive a signal from the mobile device indicating an insulin delivery adjustment amount of insulin to be delivered as determined by the artificial pancreas application. The controller may output a drive control signal to the pump mechanism to deliver the insulin delivery adjustment amount of insulin.

### BRIEF DESCRIPTION OF THE DRAWINGS

FIG. 1 provides a block diagram illustration of an example of a mobile computing device suitable for implementing an example of an early exercise detection process.

FIG. 2 illustrates a flowchart of an example process for early exercise detection.

FIG. 3 illustrates an example of a subprocess that responds to a model mismatch usable in combination with the example process of FIG. 2.

FIG. 4 illustrates another example of a subprocess of an early exercise detection application as described with reference to the examples of FIGS. 1-3.

FIG. 5 illustrates an example of a drug delivery system that utilizes one or more examples of the early exercise detection application as described with reference to the examples of FIGS. 1-4.

### DETAILED DESCRIPTION

This disclosure presents various systems, components, and methods operable to provide an indication of early exercise detection and adjust insulin delivery to a user based early detection of a user exercising and providing insulin delivery amounts of insulin that are customizable to the individual user, thus providing a more optimal solution and an improvement over systems providing limited resources to compensate for a diabetic user exercising. Each of the systems, components, and methods disclosed herein provides one or more advantages over conventional systems, components, and methods, such as an early detection of exercise, confirmation of exercise, and customizable diabetes treatment plan based on an individual's response to exercise.

An example provides a process that in which an indication early detection of an individual participating in exercise may be used with any additional algorithms or computer applications that implement a diabetes treatment plan that manages blood glucose levels and insulin therapy for a diabetic user. As discussed herein, the additional algorithms or computer application may be referred to as an “artificial pancreas” algorithm-based system, or more generally, an artificial pancreas (AP) application. An AP application may be programming code stored in a memory device and that is executable by a processor, controller or computer device, such as a smartphone, tablet, personal diabetes management device or the like. Examples of artificial pancreas (AP) application as discussed herein provide automatic delivery of an insulin based on inputs from a blood glucose sensor input, such as that received from a CGM or the like, camera imaging and object recognition, global positioning system devices, and the like.

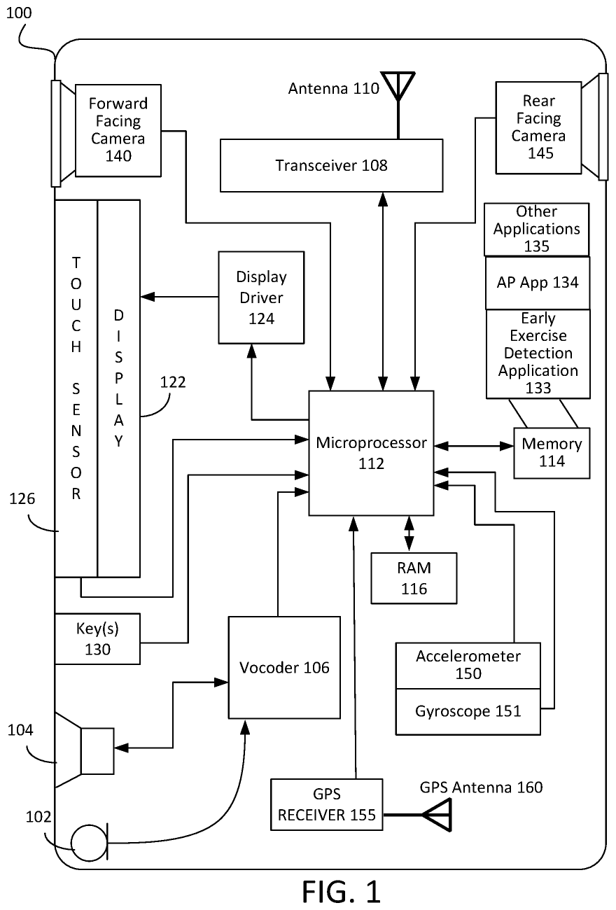

In an example, the artificial pancreas (AP) application when executed by a processor may enable a system to monitor a user's glucose values, determine an appropriate level of insulin for the user based on the monitored glucose values (e.g., blood glucose concentrations or blood glucose measurement values) and other information, such as user-provided information, such as carbohydrate intake, meal times or the like, and take actions to maintain a user's blood glucose value within an appropriate range. The appropriate blood glucose value range may be considered a target blood glucose value of the particular user. For example, a target blood glucose value may be acceptable if it falls within the range of 80 mg/dL to 140 mg/dL, which is a range satisfying the clinical standard of care for treatment of diabetes. However, an AP application as described herein may account for an activity level of a user to more precisely establish a target blood glucose value and may set the target blood glucose value at, for example, 110 mg/dL, or the like. As described in more detail with reference to the examples of FIGS. 1-5, the AP application may utilize the monitored blood glucose values and other information to generate and send a command to a wearable drug delivery device including, for example, a pump, to control delivery of insulin to the user, change the amount or timing of future doses, as well as to control other functions. In the examples, an AP application may receive a number of inputs from different systems including other devices that are not dedicated to implementing a personal diabetes treatment plan.

In an example, an early exercise detection application may operate as a plug-in to or a component of the AP application. The early exercise detection application examples may receive permission from an individual to access a camera on an individual's portable computing device (such as a smartphone, personal diabetes management device is so equipped, tablet device or the like). Examples of an individual's portable computing device may include a smartphone, a portable diabetes management device, a smartwatch, or any other portable computing device equipped with a camera. In an example, the individual's camera-equipped, portable computing device may be a smartphone upon which is installed an early exercise detection application, which is a computer application.

FIG. 1 provides a block diagram illustration of an example of a mobile computing device suitable for implementing an example of an early exercise detection process. An example of a mobile computing device may be smartphone 100. The smartphone 100 includes microphone 102, speaker 104 and vocoder 106, for audio input and output functions. The smartphone 100 also includes at least one digital transceiver (XCVR) 108, for digital wireless communications, although smartphone 100 can include additional digital or analog transceivers. For example, the smartphone 100 may include a Bluetooth® transceiver, a Wi-Fi transceiver as a well as a cellular transceiver. In the example, the transceiver 108 may provide two-way wireless communication of information, such as speech information and/or digital information by connecting with other Bluetooth-enabled devices, other Wi-Fi-enabled devices and other cellular devices as well as cellular networks and/or Wi-Fi networks via radio frequency (RF) signals to send/receive amplifiers (not separately shown) to antenna 110 or other dedicated RF protocol antennas.

The smartphone 100 may, for example, include memory 114, which may be a flash-type program memory or the like, for storage of various program routines and mobile configuration settings. Smartphone 100 may also include a random access memory (RAM) 116 for a working data processing memory. Of course, other data storage devices or configurations can be added to or substituted for those in the example.

Microprocessor 112 serves as a programmable controller for smartphone 100, in that it performs functions of smartphone 100 in accord with programming that it executes. For example, the microprocessor 112 may be operable to execute the early exercise detection application 133 and an AP application 134 in addition to other applications 135, such as a calendar application, event manager application (e.g., a personal digital assistant that manages the user's events, such as exercise schedule, or the like), a cellular telephone application, a messaging application, a fitness application, an image recognition application, a pedometer application, heart rate monitor application, or the like, that may be stored in memory 114. Both the early exercise detection application 133 and the AP application 134 may be operable to access data from the other applications 135. For example, the early exercise detection application 133 and the AP application 134 may be able to obtain calendar information from a calendar application that is one of the other applications 135. The early exercise detection application 133 may be programming code that when executing by the microprocessor 112 receives inputs from components of the smartphone 100.

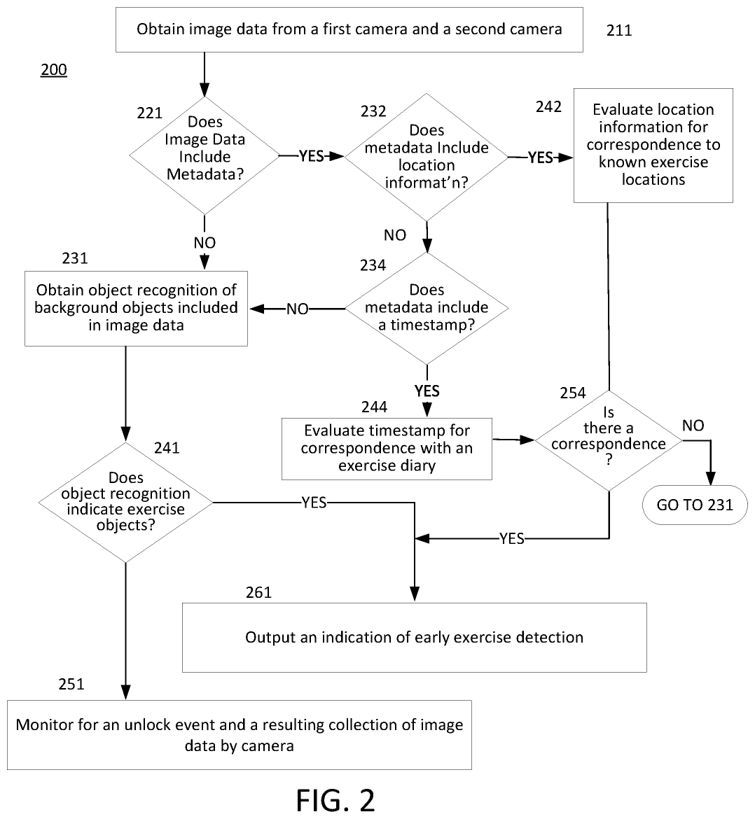

Hence, as outlined above, smartphone 100 includes a processor and programming stored in flash memory 114 that configures the processor so that the smartphone 100 is capable of performing various desired functions such as early detection of exercise as described with reference to the detailed description including the detailed examples of FIGS. 2-5.

In the example shown in FIG. 1, the user input elements for smartphone 100 include a display 122 (also referred to as “display screen 122” or “touch-screen display 122”) and can further include one or more hardware keys 130. For example, keys 130 may be implemented as a sliding keyboard containing a full alphanumeric keyboard, or may be one or more function keys, such as a home button or the like. In general, touch-screen display 122 of smartphone 100 may be used to present information (e.g., text, video, graphics or other content) to the user of the smartphone. Touch-screen display 122 may be, for example and without limitation, a capacitive touch-screen display.

Accordingly, microprocessor 112 controls touch-screen display 122 via display driver 124, to present visible outputs to the user. Touch sensor 126 may be relatively transparent, so that the user may view the information presented on the display 122. Display 122 and touch sensor 126 (and possibly one or more keys 130, if included) are the physical elements providing the textual and graphical user interface for smartphone 100. Microphone 102 and speaker 104 can be used as additional user interface elements and for audio input and output.

In the illustrated example of FIG. 1, the smartphone 100 also includes one or more digital cameras, such as digital camera 140 and 145, for capturing still images and/or video clips. In an example, the smartphone 100 may be operable to “lock” (i.e., assume a standby setting that does not permit use of the smartphone except possibly in case of an emergency) after a period of inactivity or in response to user input to lock the smartphone 100. In such an example, a user may be able to “unlock” their smartphone by entering a personal identification number (PIN) or by using a biometric input, such as a fingerprint, voice password, or facial recognition. In instances where the smartphone 100 is equipped for facial recognition and operable to unlock the smartphone using facial recognition, the digital cameras 140 and 145 may be operable to detect an image of a user as the user engages their smartphone 100 to unlock the smartphone. The smartphone 100 may further be operable, when detecting the face of the user, to obtain an image (i.e., a digital representation) of the background and detect objects in the background. Object recognition services may be provided onboard the smartphone 100 or may be available through a service accessible via a network (not shown) or cloud-based service (shown in another example). For example, see on the Internet the link to machinelearning.apple.com/2017/11/16/face-detection.html or the like. Based on the obtained image of the background, the microprocessor 112 may be operable to perform object recognition and determine whether the location of the smartphone 100 is a gym or fitness center, or not. For example, the digital cameras 140 and 145 may supply the image or digital representation of the area within its view to the microprocessor 112. The microprocessor 112 as well as other processors onboard such as a graphics processing unit (GPU) or the like (not shown separately), may process the images and detect whether any objects are recognizable in the detected images The objects to be recognized may be fitness equipment (e.g., treadmills, weightlifting machines, yoga balls or the like), objects (e.g., furniture, text, signage, doorway configuration or the like) within a lobby, foyer or entrance area of the respective gym or fitness center, or the like.

In this example, smartphone 100 also includes one or more motion sensors, such as accelerometer 150 or gyroscope 151 for detecting motion of the smartphone in response to an individual's movements. Examples of motion sensors include an accelerometer and/or gyroscope and associated circuitry for signaling microprocessor 112 in response to detected motion input. The detected motion input may include, for example, a change in orientation of the physical device within three-dimensional space, as described above, as well as a determined rate of change in position of the smartphone 100. In this way, smartphone 100 can use the accelerometer 150 or the gyroscope 151 to monitor and track the detected motion or physical movement. The tracked motion detected by accelerometer 150 or gyroscope 151 can be used by microprocessor 112 to determine whether the rate of such movement corresponds to a pattern of movement associated with indicators of early stages of exercise (e.g., beginning movements, such as stretches or warmup routines).

The smartphone 100 may also be equipped with a global positioning system (GPS) receiver 155 that is operable to receive GPS signals via a GPS antenna 160. The GPS receiver 155 may be communicatively coupled to the microprocessor 112. The microprocessor 112 may use location information provided by the GPS receiver 155 to provide location information. In the example, the microprocessor 112 may be operable to determine a location of the smartphone 100 based on signals received from the global positioning system receiver 155 or the Wi-Fi transceiver 108.

As mentioned, the early exercise detection application 133 and the AP application 134 may be stored in the memory 114 of the smartphone 100. The early exercise detection application 133 may be programming code developed to enable the smartphone 100 to provide an indication that the individual is about to or is exercising (or about to exercise) as an input to an AP application 134. In some examples, the AP application 134 may include the functionality provided by the early exercise detection application 133 such that a separate application (i.e., early exercise detection application 133). For example, the early exercise detection application 133 may use various information provided by hardware and/or software components of the smartphone 100 to provide an early detection of exercise. For example, the early exercise detection application 133 may use camera information to detect exercise, and sensors that may indicate the presence of perspiration (above what may be set as normal levels) may be used to provide an early indication of exercise, or when different models for delivering insulin dosages to a user experience a mismatch that is outside a mismatch threshold, or data from other combinations of multiple sensors or mobile device components (e.g., accelerometers, barometers, location services, or the like) that may be used to detect exercise.

In an example, the early exercise detection application 133 may be granted permission by the user to access the images (which may also be referred to as image data or digital representations) generated by cameras 140 and 145 when unlocking the smartphone 100 using the facial recognition features of the smartphone 100.

The early exercise detection application 133 may perform an example of a process and/or subprocesses as shown in the examples of FIGS. 2-5 that allow for an early detection of exercise and determine an optimal insulin delivery amount to be delivered to a user in response to changes in insulin requirements due to the user participation in exercise. The examples enable improved and personalized adjustment and modification to the amount of insulin to be delivered and the timing of the delivery of the adjusting insulin delivery amount.

FIG. 2 illustrates a flowchart of an example process for early exercise detection. As mentioned, the mobile device such as smartphone 100, may have settings that allow the cameras of the mobile device to collect image data when the mobile device is executing an unlock procedure. While executing the unlock procedure, the mobile device may be operable to execute an early exercise application which utilizes a process of obtaining image data from a camera coupled to the processor (211). For example, the smartphone 100 may be operable to activate one of cameras 140 and 145 or both cameras 140 and 145 during an unlock procedure, when early exercise detection application is active. The early detection application may be active, when the user opens and launches the early exercise detection application by positively selecting it in a graphical user interface to open and launch on the smartphone 100, or if the user provides permission for the early exercise detection application to operate in the background in which case the early exercise detection application opens and launches when the smartphone 100 is powered ON. In the example, the processor may, obtain, during an unlock procedure or immediately before or after, first image data from a first camera, which may be a forward facing camera (where forward facing is toward a user). Depending upon settings in the exercise detection application, the processor may also be operable to obtain, during an unlock procedure or immediately before (e.g., a user depresses the home key to access their mobile device, but the mobile device operating system indicates the mobile device is locked), second image data from a second camera, which may be a rear facing camera (where rear facing is away from a user).

The smartphone 100 may be operable to obtain image data (i.e., an image comprising pixel information such as luminance, brightness, saturation, hue, or color information, such as RGB, HSV, CIE, or the like) from the camera. The image data may, for example, include metadata. In an example, the metadata may include location information or timestamp information.

At 221, the processor may determine whether any of the obtained image data includes metadata. If the response is YES, the process 200 may proceeds to 232. At 232, the processor may determine whether the metadata of the obtained image data includes location information. If the response at 232, is NO, the process 200 may proceed to 234 at which the processor may determine whether the metadata of the obtained image data includes timestamp information.

Returning to step 232, the processor, based on a determination that the metadata includes location information, may evaluate, at 242, the location information for a correspondence to known exercise locations. For example, the processor may have access to an exercise diary. An example of an exercise diary may be a calendar of exercise appointments, notes from exercise on a particular day, or the like.

Alternatively, in response to a determination that the metadata includes timestamp information, the processor, at 244, may evaluate the timestamp information for a correspondence of a time stamp in the exercise diary. At 254, the processor may identify a correspondence with either an exercise location included in the known exercise locations (from 242) or an exercise time in the exercise diary (from 244).

In another example of identifying a correspondence with an exercise time reservation, the processor may be operable to perform functions to access an event manager; and identify events and exercise that a user has scheduled participation that correspond to the timestamp information from the metadata of the image data. Based on the identified events and exercise the user has scheduled participation corresponding to the timestamp information, the process 200 executed by the processor may proceed to step 261. At 261, the processor may output an indication of early exercise detection to the artificial pancreas application. The artificial pancreas application may respond to the indication of early exercise detection by modifying insulin delivery to the user for a period of time, such as 30 minutes, an hour or several hours.

In another example, the processor may be operable to identify a correspondence with an exercise location in the known exercise locations by performing functions to obtain, via an input from a user interface, a name of the exercise location and a confirmation of a global positioning system indication of the exercise location; and store the obtained name of the exercise location in the table of known exercise locations. Based on the identified correspondence with an exercise location in the known exercise locations, the process 200 executed by the processor may proceed to step 261. At 261, the processor may output an indication of early exercise detection to an artificial pancreas application. The artificial pancreas application may respond to the indication of early exercise detection by modifying insulin delivery to the user for a period of time, such as 30 minutes, an hour or several hours.

Alternatively, at 254, the processor may determine that a correspondence between the timestamp information from the metadata of the image and scheduled participation in exercise cannot be identified or determine that a correspondence between the location information from the metadata of the image and a location of the mobile device at a known exercise location cannot be identified.

In response to a correspondence not being identified at 254, the process 200 may proceed to 231. At 231, the first image data obtained from a first camera, and, if available, the second image data obtained from a second camera, may be submitted to an object recognition process. The object recognition process may be implemented by a mobile computer application stored in a memory of the mobile device, or by an external service that is accessible by the mobile device via a transceiver and a network connection. Whether the image recognition process is implemented by the mobile computer application executed on the mobile device or by the external service, the processor via execution of the early exercise detection application may be operable to receive an indication from the object recognition process that exercise-related objects are present in either the obtained first image data or the obtained second image data. In an example, the processor may access a table of known exercise locations that includes exercise object data to identify a correspondence between an exercise-related object and a known exercise location to confirm that the presence of the exercise-related objects. At 241, the processor may determine a percentage of correspondence between the recognized objects from the image data and known exercise locations. Based on the percentage of correspondence between the recognized object and known exercise, the early exercise detection application may generate a confidence level indicating a probability of a detection of exercise. The confidence may be used in the determination of the insulin delivery adjustment amount of insulin.

Using the indication of exercise-related objects, the processor may be operable to receive an input from a location service executing on the mobile device indicating a location of the mobile device and by association the location of a wearable drug delivery device. The processor may obtain location information related to the exercise, such as locations of fitness centers, ballparks, gymnasiums, competitive race locations (e.g. Pittsburgh or Boston Marathon), or the like. The processor may be operable to evaluate, which may be a comparison or the like, the received input indicating the location of the wearable drug delivery device and the obtained location information related to exercise. Based on a result of the evaluation indicating that exercise is probably occurring or going to occur, the processor may be operable to alter the insulin delivery adjustment amount. For example, the early exercise detection application may provide an input to an artificial pancreas application, which may be operable to respond to the input from the early exercise detection application by adjusting insulin delivery to the user during a period of time, such as 1 hour, 2 hours or longer.

Alternatively, in response to a correspondence not being identified at 241, the process 200 may proceed to 251 during which the processor may be operable to monitor the mobile device for an unlock event to collect image data from a camera. For example, the processor may determine a time has expired and the processor has locked the mobile device, or that the user has chosen to lock the mobile device (for example, the user may be driving or has placed to mobile device down), and the processor may monitor a home button, microphone or other buttons of the mobile device used to input an open or unlock sequence.

Other sensors may also be suitable for providing information data usable to determine detect at an initial onset of exercise and generate an early exercise detection indication. The smartphone 100 may be operable to detect various motion parameters (e.g., acceleration, deceleration, speed, orientation, such as roll, pitch, yaw, compass direction, or the like) that may be indicative of the activity of the user. For example, the sensors may output signals in response to detecting motion of the mobile device that may be interpreted by the early exercise detection application as indicative of exercise. Based on the received signals, the processor under control of the early exercise detection application may cause the artificial pancreas application to adjust operation related to drug delivery, for example, by adjusting an amount of insulin to be delivered to a user for a period of time. The period of time may be a predetermined period of time, such as 1 hour, 2 hours or the like, or may be until there is an indication that the user's blood glucose is rising (as determined based on blood glucose measurements received from a continuous blood glucose monitor or input from a user (by a guardian or physician) who measures the user's blood glucose.

In a specific example, a process for determining whether a user is participating in exercise may utilize a corresponding change capacitance in response to the user perspiring. In the example, the processor may be communicatively coupled to a wearable accessory, a continuous blood glucose monitor (referred to as a CGM), a wearable drug delivery device, or another device coupled to the skin of a user. The CGM, a wearable drug delivery device, or another device coupled to the skin of a user device may include a pair of electrodes coupled to the user and respective circuitry to enable the application of a voltage or current to an electrode of the pair. The processor may be operable to detect a first electrical property between a pair of electrodes coupled to a user.

In a more detailed example, a first electrode and a second electrode of the pair of electrodes may be positioned a predetermined distance apart. The predetermined distance apart may be a distance substantially equal to a distance on the bottom and from opposite ends of a continuous blood glucose monitor or a wearable drug delivery device. The processor may be operable to determine a first electrical property (e.g., a voltage, a current, a resistance, a capacitance, or the like) between the pair of electrodes coupled to a user. After a period of time has elapsed, the processor may be operable to determine a second electrical property between the pair of electrodes. The processor may be operable to determine a difference between the first determined electrical property and the second determined electrical property. The difference may, for example, be due to perspiration of the user or another condition that effects the electrical property. The processor may be operable to determine that the difference corresponds to values of previously determined differences associated with a user exercising that have been stored in a user history database. For example, the values of previously determined differences may correspond to periods of known exercise by the user. In response to the processor determining the correspondence between the previously determined differences and periods of known exercise by the user, the processor may be operable to generate and output a signal confirming the indication of early exercise detection.

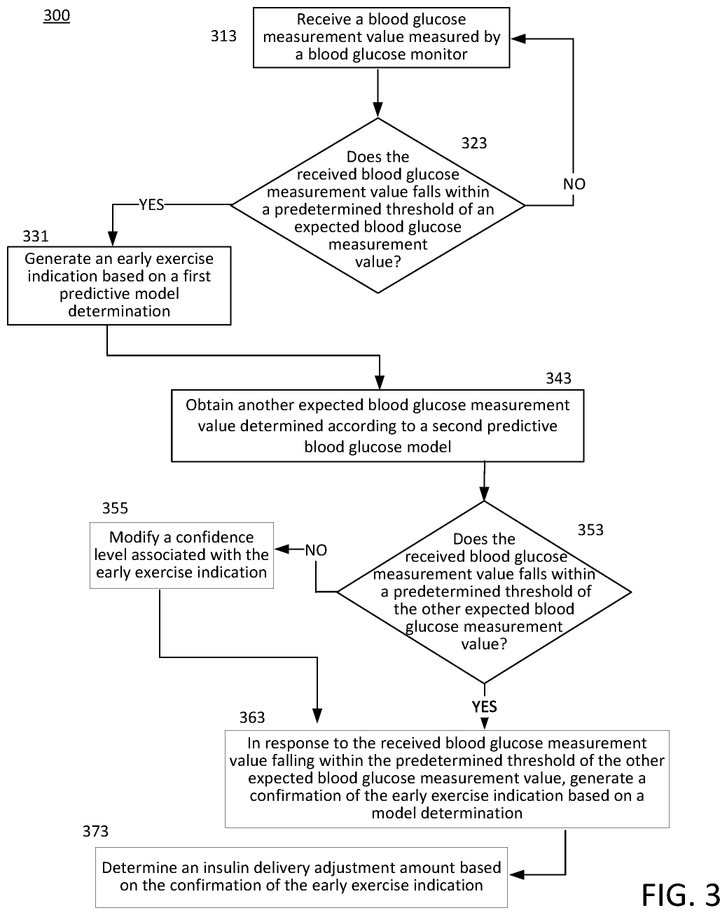

In another example, the processor may execute a process such as that illustrated in FIG. 3. FIG. 3 illustrates an example of a process that responds to a model mismatch usable in combination with the example process of FIG. 2. The process 300 may be implemented in programming code contained within the early exercise detection application to determine a correspondence between an indication of early exercise detection and sensor data, such as a blood glucose measurement value based on a measurement provided by a blood glucose sensor as shown in. The process 300 when executed by the processor may cause the processor to be operable to receive a blood glucose measurement value from a blood glucose monitor (313). At 323, the processor may determine whether the received blood glucose measurement value falls within a predetermined threshold of an expected blood glucose measurement value. The predetermined threshold may, for example, be a tolerance that extends from below a blood glucose measurement value set point (e.g., between 110 to 150 mg/dL or the like) to over the blood glucose measurement value set point (e.g., between 110 to 150 mg/dL or the like). The predetermined threshold may be set as a plus or minus (±) 10 (mg/dL), (±) 20 (mg/dL), as a percentage difference, or the like. For example, the processor may determine or calculate the expected blood glucose measurement value according to a first predictive blood glucose model. This first predictive blood glucose model may be formulated as a recursive model of past insulin and glucose values, as follows:

G′[k]=K1′[k−3]+b₁G′[k−1]+b₂G′[k−2]+b₃G′[k−3]

where G[k] is the k^{th} predicted glucose value, I[k] is the k^{th} insulin delivery value, and bₙ are tunable parameters.

In response to the processor determining the received blood glucose measurement value falls outside the predetermined threshold of the expected blood glucose measurement value calculated or determined using the first predictive blood glucose model at 323, the processor responds by returning to step 313 to monitor for receipt of a next blood glucose measurement value.

In response to the processor determining the received blood glucose measurement value falls within the predetermined threshold of the expected blood glucose measurement value calculated or determined using the first predictive blood glucose model at 323, the processor may generate an indication of early exercise detection based on a model determination (331). In addition to generating the indication of early exercise detection, the processor may also generate a confidence level.

After 331, the processor executing the early exercise detection application may be operable, in response to generating the indication of early exercise detection based on the first model determination, to obtain another expected blood glucose measurement value determined according to a second predictive blood glucose model (343). The second predictive blood glucose model may calculate a second expected blood glucose measurement value using inputs or coefficients determined from data including senor data that is different from the inputs or coefficients used by the first predictive blood glucose model. For instance, if the first predictive model utilizes past glucose and insulin delivery values, the second predictive model may instead utilize glucose and insulin onboard (IOB) values instead, as follows:

G′[k]=KIOB′[k−3]+b₁G′[k−1]+b₂G′[k−2]+b₃G′[k−3]

where IOB[k] is the k^{th} IOB value.

Alternately, the first predictive blood glucose model may utilize a model based on insulin onboard (JOB) to calculate a first expected blood glucose measurement value and the second predictive blood glucose model may utilize total daily insulin (TDI) to calculate a second expected blood glucose measurement value.

At 353, the processor may be further operable to determine whether the received blood glucose measurement value falls within a predetermined threshold of the other expected blood glucose measurement value generated according to the second predictive blood glucose model. In response to the received blood glucose measurement value falling within the predetermined threshold of the other expected blood glucose measurement value, the processor may generate a confirmation of the indication of early exercise detection based on the other (e.g., second) model determination (363).

In this example and as mentioned previously, the early exercise detection application may function as a plug-in to the artificial pancreas application and, as a result, may, in different examples, function alone, together with the artificial pancreas application, or even cooperate with an example of an AID system provided by a third-party, to provide the indication of early exercise detection and the determination of the insulin delivery adjustment amount as well as instructing a drug delivery device to deliver the insulin delivery adjustment amount. For example, the early detection application may not be tied to any other specific algorithm or pump and may be utilized in any system.

The generated confirmation may be output and received by an artificial pancreas application that may also be executed by the processor. The artificial pancreas application may be operable to determine an insulin delivery adjustment amount based on the confirmation of the indication of early exercise detection (373). For example, the artificial pancreas application may have preset insulin delivery adjustment amounts that are used in response to an early exercise determination indication. The preset insulin delivery adjustment amounts may be based, for example, on age, weight, time of day or the like, so as to allow nearly universal application of the preset insulin delivery adjustment amounts to users of the early exercise detection application. The settings that are not customized by only based on age, weight, and/or time of day may be considered default settings. Alternatively, the artificial pancreas application may be operable to calculate a customized insulin delivery adjustment amount for delivery to users of the early exercise detection application. The processor when calculating the customized insulin delivery may consider in addition to age, weight, and/or time of day, may also utilize insulin delivery history, blood glucose measurement value history, history of participating in exercise (e.g., a fitness class every Thursday at 9 AM), or the like.

In a further example, in response to the indication of early exercise detection generated at 331, the processor may determine a confidence level of a user's expected participation in exercise. The confidence level may be generated, for example, by an algorithm that utilizes a history of past blood glucose measurement values, past user inputs indicating exercise, a relative closeness of a received blood glucose measurement value to a respective expected blood glucose measurement value (e.g., the received blood glucose measurement value is within 10% or 5% of the expected blood glucose measurement values, or the like). The processor may be operable to assign a confidence level based on the relative closeness. For example, the processor may assign a 65% confidence level when the received blood glucose measurement value to expected blood glucose measurement value is within 10%. Alternatively, the processor may assign an 80% confidence level when the received blood glucose measurement value to expected blood glucose measurement value is within 5%. Of course, different confidence levels may be assigned based on user history or from the history of a number of users of the early exercise detection application. Continuing with the example, at 373, the processor may determine, based on the determined confidence level, an insulin delivery adjustment amount for a next delivery of insulin, and output instructions to deliver the determined insulin delivery adjustment amount.

Returning to the example at step 353, when the received blood glucose measurement value does not fall within the predetermined threshold of the other expected blood glucose measurement value determined using the second predictive blood glucose model, the process may proceed to 355. At 355, the processor may be operable to modify a confidence level associated with the indication of early exercise detection. As mentioned, the confidence level may be generated, at 331, by an algorithm that utilizes a history of past blood glucose measurement values, past user inputs indicating exercise, a relative closeness of a received blood glucose measurement value to a respective expected blood glucose measurement value (e.g., the received blood glucose measurement value is within 10% or 5% of the expected blood glucose measurement values, or the like). For example, at 355, the processor may modify the confidence level associated with the indication of early exercise detection. The modification of the confidence level at 355 may be a modification decreasing the confidence level due to the failure of the received blood glucose measurement value to be within a threshold of the respective expected blood glucose measurement value. However, in some examples, sources of confirmation other than the first predictive blood glucose model may be used that result in the confidence level being increased after the other sources of confirmation (or correspondence) are considered.

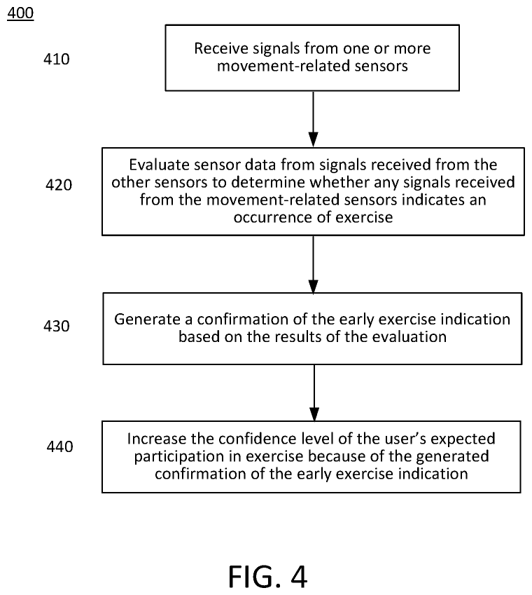

Other processes for confirming an indication of early exercise detection are also contemplated. FIG. 4 illustrates another example of a subprocess of an early exercise detection application as described with reference to the examples of FIGS. 1-3. The process 400 may be performed after step 261 of the process example shown in FIG. 2. In the other example, the early exercise detection application executed by the processor may before, during or after generating the receive signals from one or more movement-related sensors (410) coupled to the processor, such as an accelerometer, a gyroscope, a barometer, a heartrate sensor, or the like. The respective sensors may be communicatively coupled to the processor. At 420, the processor when executing the early exercise detection application may be operable to evaluate sensor received from the other sensors to determine whether any signals received from the movement-related sensors indicates an occurrence of exercise. For example, the accelerometer and/or gyroscope may begin outputting signals indicative of rapid lateral movement of the mobile device, which may be indicative of running or jumping. In response to a determination of an indication of exercise, the processor executing the early exercise detection application may be operable to generate a confirmation of the early exercise indication (430).

The processor, at 440, may increase the confidence level of the user's expected participation in exercise because of the generated confirmation of the early exercise indication. In an example, the early exercise detection application may provide the generated confirmation and the confidence level to the artificial pancreas application for use in calculating an insulin delivery adjustment amount.

Alternatively, if the evaluation by the processor at 420 determines that the received signals are not indicative of an occurrence of exercise, the processor may not generate a confirmation of an early detection of exercise. In which case, the process 400 may return to 410 to receive updated or subsequent signals from the one or more movement-related sensors.

The process 400 may be used as supplemental process to confirm the indication of early exercise detection output from process 200, step 261. For example, the AP application may have established an insulin delivery adjustment amount in response to the output at step 261 of process 200 but may modify that established insulin delivery adjustment amount based on the increase in the confidence level of the user's expected participation. The AP application may be further operable to and output instructions to deliver a modified insulin delivery adjustment amount instead of the determined insulin delivery adjustment amount. The outputted instructions may be delivered to and actuate a wearable drug delivery device to deliver the modified insulin delivery adjustment amount to a user.

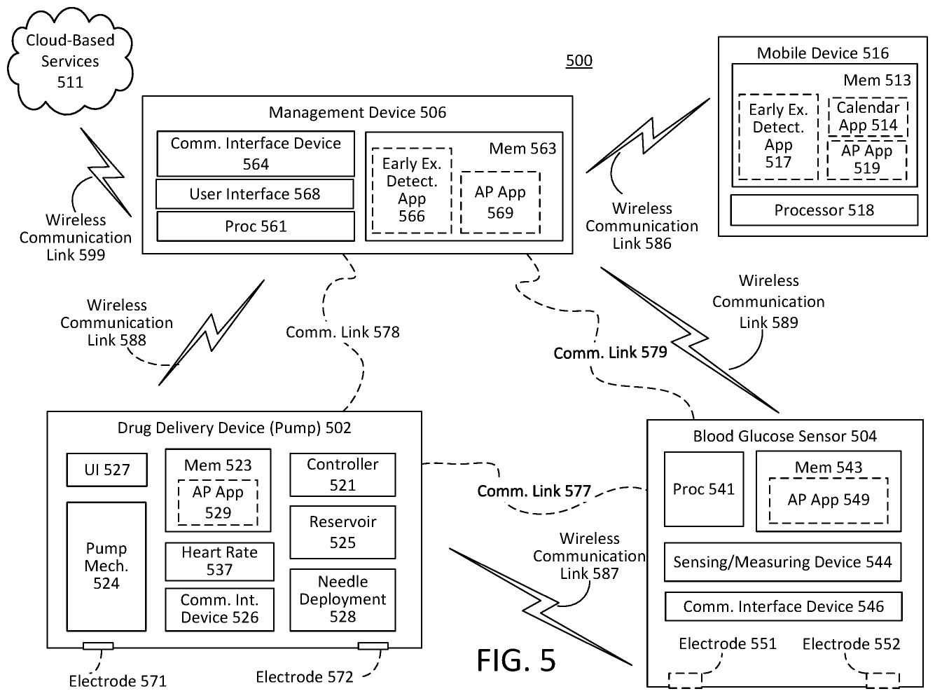

FIG. 5 illustrates an example of a drug delivery system that utilizes one or more examples of the early exercise detection application as described with reference to the examples of FIGS. 1-4. The drug delivery system 500 may include a drug delivery device 502, a management device 506, and a blood glucose sensor 504.

In the example of FIG. 5, the drug delivery device 502 may be a wearable or on-body drug delivery device that is worn by a patient or user on the body of the user. As shown in FIG. 5, the drug delivery device 502 may include a pump mechanism 524 that may, in some examples be referred to as a drug extraction mechanism or component, and a needle deployment component 528. In various examples, the pump mechanism 524 may include a pump or a plunger (not shown).

The needle deployment component 528 may, for example include a needle (not shown), a cannula (not shown), and any other fluid path components for coupling the stored liquid drug in the reservoir 525 to the user. The cannula may form a portion of the fluid path component coupling the user to the reservoir 525. After the needle deployment component 528 has been activated, a fluid path (not shown) to the user is provided, and the pump mechanism 524 may expel the liquid drug from the reservoir 525 to deliver the liquid drug to the user via the fluid path. The fluid path may, for example, include tubing (not shown) coupling the wearable drug delivery device 502 to the user (e.g., tubing coupling the cannula to the reservoir 525).

The wearable drug delivery device 502 may further include a controller 521 and a communications interface device 526. The controller 521 may be implemented in hardware, software, or any combination thereof. The controller 521 may, for example, be a processor, a logic circuit or a microcontroller coupled to a memory. The controller 521 may maintain a date and time as well as other functions (e.g., calculations or the like) performed by processors. The controller 521 may be operable to execute an artificial pancreas algorithm stored in the memory that enables the controller 521 to direct operation of the drug delivery device 502. In addition, the controller 521 may be operable to receive data or information indicative of the exercise of the user from the mobile device, as well as from any other sensors (such as those (e.g., accelerometer, location services application or the like) on the management device 506 or CGM 504) of the drug delivery device 502 or any sensor coupled thereto, such as a global positioning system (GPS)-enabled device or the like.

The controller 521 may process the data from the mobile device or any other coupled sensor to determine if an alert or other communication is to be issued to the user and/or a caregiver of the user or if an operational mode of the drug delivery device 502 is to be adjusted. The controller 521 may provide the alert, for example, through the communications interface device 526. The communications interface device 526 may provide a communications link to one or more management devices physically separated from the drug delivery device 502 including, for example, a management device 506 of the user and/or a caregiver of the user (e.g., a parent). The communication link provided by the communications interface device 526 may include any wired or wireless communication link operating according to any known communications protocol or standard, such as Bluetooth or a cellular standard.

The example of FIG. 5 further shows the drug delivery device 502 in relation to a blood glucose sensor 504, which may be, for example, a continuous glucose monitor (CGM). The CGM 504 may be physically separate from the drug delivery device 502 or may be an integrated component thereof. The CGM 504 may provide the controller 521 with data indicative of measured or detected blood glucose (BG) levels of the user.

The management device 506 may be maintained and operated by the user or a caregiver of the user. The management device 506 may control operation of the drug delivery device 502 and/or may be used to review data or other information indicative of an operational status of the drug delivery device 502 or a status of the user. The management device 506 may be used to direct operations of the drug delivery device 502. For example, the management device 506 may be a dedicated personal diabetes management (PDM) device, a smartphone, a tablet computing device, other consumer electronic device including, for example, a desktop, laptop, or tablet, or the like. The management device 506 may include a processor 561 and memory devices 563. The memory devices 563 may store an early exercise detection application 566 as discussed with reference to the examples of FIGS. 1-4 as well as an artificial pancreas application 569 including programming code that may implement delivery of insulin based on input from the early exercise detection application. The early exercise detection application 566 may be operable to receive inputs from various devices such the heart rate monitor 537 or sensing/measuring device 544.

The management device 506 may receive alerts, notifications, or other communications from the drug delivery device 502 via one or more known wired or wireless communications standard or protocol.

In an example, the management device 506 may operate in cooperation with a mobile device 516. The mobile device 516 is shown with a memory 513 and a processor 516 but may also include additional components and elements as discussed with reference to smartphone 100 of FIG. 1. The memory 513 may store programming code as well as mobile computer applications, such as the early exercise detection application 517 and an artificial pancreas (AP) application 519.

The drug delivery system 500 may be operable to implement an AP application, such as 519, 569 or 529 that includes functionality to determine a movement of a wearable drug delivery device that is indicative of exercise of the user, implement an activity mode, a hyperglycemia mode, a hypoglycemia mode, and other functions, such as control of the wearable drug delivery device. The drug delivery system 500 may be an automated drug delivery system that may include a wearable drug delivery device (pump) 502, a sensor 504, and a personal diabetes management device (PDM) 506.

In an example, the wearable drug delivery device 502 may be attached to the body of a user, such as a patient or diabetic, and may deliver any therapeutic agent, including any drug or medicine, such as insulin or the like, to a user. The wearable drug delivery device 502 may, for example, be a wearable device worn by the user. For example, the wearable drug delivery device 502 may be directly coupled to a user (e.g., directly attached to a body part and/or skin of the user via an adhesive or the like). In an example, a surface of the wearable drug delivery device 502 may include an adhesive to facilitate attachment to a user.

The wearable drug delivery device 502 may frequently be referred to as a pump, or an insulin pump, in reference to the operation of expelling a drug from the reservoir 525 for delivery of the drug to the user.

In an example, the wearable drug delivery device 502 may include a reservoir 525 for storing the drug (such as insulin), a needle or cannula (not shown) for delivering the drug into the body of the user (which may be done subcutaneously, intraperitoneally, or intravenously), and a pump mechanism (mech.) 524, or other drive mechanism, for expelling the stored insulin from the reservoir 525, through a needle or cannula (not shown), and into the user. The reservoir 525 may be operable to store or hold a liquid or fluid, such as insulin, morphine, or another therapeutic drug. The pump mechanism 524 may be fluidly coupled to reservoir 525, and communicatively coupled to the controller 521. The wearable drug delivery device 502 may also include a power source (not shown), such as a battery, a piezoelectric device, or the like, for supplying electrical power to the pump mechanism 524 and/or other components (such as the controller 521, memory 523, and the communication interface device 526) of the wearable drug delivery device 502. Although also not shown, an electrical power supply for supplying electrical power may similarly be included in each of the sensor 504, the smart accessory device (if present), and the management device (PDM) 506.

In an example, the blood glucose sensor 504 may be a device communicatively coupled to the processor 561 or 521 and may be operable to measure a blood glucose value at a predetermined time interval, such as approximately every 5 minutes, or the like. The blood glucose sensor 504 may provide a number of blood glucose measurement values to the AP applications operating on the respective devices. For example, the blood glucose sensor 504 may be a continuous blood glucose sensor that provides blood glucose measurement values to the AP applications operating on the respective devices periodically, such as approximately every 5, 10, 12 minutes, or the like.

The wearable drug delivery device 502 may when operating in a normal mode of operation may provide insulin stored in reservoir 525 to the user based on information (e.g., blood glucose measurement values, inputs from an inertial measurement unit, global positioning system-enabled devices, Wi-Fi-enabled devices, or the like) provided by the sensor 504 and/or the management device (PDM) 506.

For example, the wearable drug delivery device 502 may contain analog and/or digital circuitry that may be implemented as a controller 521 (or processor) for controlling the delivery of the drug or therapeutic agent. The circuitry used to implement the controller 521 may include discrete, specialized logic and/or components, an application-specific integrated circuit, a microcontroller or processor that executes software instructions, firmware, programming instructions or programming code (enabling, for example, the artificial pancreas application (AP App) 529 as well as the process examples of FIGS. 5-6B) stored in memory 523, or any combination thereof. For example, the controller 521 may execute a control algorithm, such as an artificial pancreas application 529, and other programming code that may make the controller 521 operable to cause the pump to deliver doses of the drug or therapeutic agent to a user at predetermined intervals or as needed to bring blood glucose measurement values to a target blood glucose value. The size and/or timing of the doses may be programmed, for example, into an artificial pancreas application 529 by the user or by a third party (such as a health care provider, wearable drug delivery device manufacturer, or the like) using a wired or wireless link, such as 520, between the wearable drug delivery device 502 and a management device 506 or other device, such as a computing device at a healthcare provider facility. In an example, the pump or wearable drug delivery device 502 is communicatively coupled to the processor 561 of the management device via the wireless link 520 or via a wireless communication link, such as 589 or wired communication link, such as 579, from the sensor 504. The pump mechanism 524 of the wearable drug delivery device may be operable to receive an actuation signal from the processor 561, and in response to receiving the actuation signal and expel insulin from the reservoir 525 and the like.

The devices in the system 500, such as management device 506, wearable drug delivery device 502, and sensor 504, may also be operable to perform various functions including controlling the wearable drug delivery device 502. For example, the management device 506 may include a communication interface device 564, a processor 561, and a management device memory 563. The management device memory 563 may store an instance of the AP application 569 that includes programming code, that when executed by the processor 561 provides the process examples described with reference to the examples of FIGS. 1-4. The management device memory 563 may also store programming code for providing the process examples described with reference to the examples of FIGS. 1-4.

Although not shown, the system 500 may include a smart accessory device may be, for example, an Apple Watch®, other wearable smart device, including eyeglasses, provided by other manufacturers, a global positioning system-enabled wearable, a wearable fitness device, smart clothing, or the like. Similar to the management device 506, the smart accessory device (not shown) may also be operable to perform various functions including controlling the wearable drug delivery device 502. For example, the smart accessory device may include a communication interface device, a processor, and a memory. The memory may store an instance of the AP application that includes programming code for providing the process examples described with reference to the examples of FIGS. 1 and 3-6B. The memory may also store programming code and be operable to store data related to the AP application.

The sensor 504 of system 500 may be a continuous glucose monitor (CGM) as described above, that may include a processor 541, a memory 543, a sensing or measuring device 544, and a communication interface device 546. The memory 543 may store an instance of an AP application 549 as well as other programming code and be operable to store data related to the AP application 549. The AP application 549 may also include programming code for providing the process examples described with reference to the examples of FIGS. 1-4.

Instructions for determining the delivery of the drug or therapeutic agent (e.g., as a bolus dosage) to the user (e.g., the size and/or timing of any doses of the drug or therapeutic agent) may originate locally by the wearable drug delivery device 502 or may originate remotely and be provided to the wearable drug delivery device 502. In an example of a local determination of drug or therapeutic agent delivery, programming instructions, such as an instance of the artificial pancreas application 529, stored in the memory 523 that is coupled to the wearable drug delivery device 502 may be used to make determinations by the wearable drug delivery device 502. In addition, the wearable drug delivery device 502 may be operable to communicate via the communication interface device 526 and wireless communication link 588 with the wearable drug delivery device 502 and with the blood glucose sensor 504 via the communication interface device 526 and wireless communication link 589.

Alternatively, the remote instructions may be provided to the wearable drug delivery device 502 over a wired or wireless link by the management device (PDM) 506. The PDM 506 may be equipped with a processor 561 that may execute an instance of the artificial pancreas application 569, if present in the memory 563. The wearable drug delivery device 502 may execute any received instructions (originating internally or from the management device 506) for the delivery of insulin to the user. In this way, the delivery of the insulin to a user may be automated.

In various examples, the wearable drug delivery device 502 may communicate via a wireless communication link 588 with the management device 506. The management device 506 may be an electronic device such as, for example, a smartphone, a tablet, a dedicated diabetes therapy management device, or the like. Alternatively, the management device 506 may be a wearable wireless accessory device, such as a smart watch, or the like. The wireless links 587-589 may be any type of wireless link provided by any known wireless standard. As an example, the wireless links 587-589 may enable communications between the wearable drug delivery device 502, the management device 506 and sensor 504 based on, for example, Bluetooth®, Wi-Fi®, a near-field communication standard, a cellular standard, or any other wireless optical or radio-frequency protocol.

The sensor 504 may also be coupled to the user by, for example, adhesive or the like and may provide information or data on one or more medical conditions and/or physical attributes of the user. The information or data provided by the sensor 504 may be used to adjust drug delivery operations of the wearable drug delivery device 502. For example, the sensor 504 may be a glucose sensor operable to measure blood glucose and output a blood glucose value or data that is representative of a blood glucose value. For example, the sensor 504 may be a glucose monitor that provides periodic blood glucose measurements a continuous glucose monitor (CGM), or another type of device or sensor that provides blood glucose measurements.

The sensor 504 may include a processor 541, a memory 543, a sensing/measuring device 544, and communication interface device 546. The communication interface device 546 of sensor 504 may include a radio-frequency transmitter, receiver, and/or transceiver for communicating with the management device 506 over a wireless communication link 589 or wired communication link 579 or with wearable drug delivery device 502 over the wireless communication link 587, or via wired communication link 577. The sensing/measuring device 544 may include one or more sensing elements, such as a blood glucose measurement element, a heart rate monitor, a blood oxygen sensor element, or the like. The processor 541 may include discrete, specialized logic and/or components, an application-specific integrated circuit, a microcontroller or processor that executes software instructions, firmware, programming instructions stored in memory (such as memory 543), or any combination thereof. For example, the memory 543 may store an instance of an AP application 549 that is executable by the processor 541.

Although the sensor 504 is depicted as separate from the wearable drug delivery device 502, in various examples, the sensor 504 and wearable drug delivery device 502 may be incorporated into the same unit. That is, in one or more examples, the sensor 504 may be a part of the wearable drug delivery device 502 and contained within the same housing of the wearable drug delivery device 502 (e.g., the sensor 504 may be positioned within or embedded within the wearable drug delivery device 502). Glucose monitoring data (e.g., measured blood glucose values) determined by the sensor 504 may be provided to the wearable drug delivery device 502 and/or the management device 506, which may use the measured blood glucose values to determine movement of the wearable drug delivery device indicative of exercise of the user, an activity mode, a hyperglycemia mode and a hyperglycemia mode.

In an example, the management device 506 may be a personal diabetes manager. The management device 506 may be used to program or adjust operation of the wearable drug delivery device 502 and/or the sensor 504. The management device 506 may be any portable electronic device including, for example, a dedicated controller, such as processor 561, a smartphone, or a tablet. In an example, the management device (PDM) 506 may include a processor 561, a management device memory 563, and a communication interface device 564. The management device 506 may contain analog and/or digital circuitry that may be operable as a processor 561 (or controller) for executing processes to manage a user's blood glucose levels and for controlling the delivery of the drug or therapeutic agent to the user. The processor 561 may also be operable to execute programming code stored in the management device memory 563. For example, the management device memory 563 may be operable to store an artificial pancreas application 569 that may be executed by the processor 561. The processor 561 may when executing the artificial pancreas application 569 may be operable to perform various functions, such as those described with respect to the examples in FIGS. 1-4. The communication interface device 564 may be a receiver, a transmitter, or a transceiver that operates according to one or more radio-frequency protocols. For example, the communication interface device 564 may include a cellular transceiver and a Bluetooth transceiver that enables the management device 506 to communicate with a data network via the cellular transceiver and with the sensor 504 and the wearable drug delivery device 502. The respective transceivers of communication interface device 564 may be operable to transmit signals containing information useable by or generated by the AP application or the like. The communication interface devices 526 and 546 of respective wearable drug delivery device 502 and sensor 504, respectively, may also be operable to transmit signals containing information useable by or generated by the AP application or the like.

The wearable drug delivery device 502 may communicate with the sensor 504 over a wireless communication link 587 (or wired communication link 577) and may communicate with the management device 506 over a wireless link 520. The sensor 504 and the management device 506 may communicate over a wireless link 588. The mobile device may communicate with the wearable drug delivery device 502, the sensor 504 and the management device 506 over wireless communication links 586, 587, 588 and 589, respectively. The wireless communication links 586, 587, 588 and 589 may be any type of wireless communication link operating using known wireless radio-frequency standards or proprietary standards. As an example, the wireless communication links 586, 587, 588 and 589 may provide communication links based on Bluetooth®, Wi-Fi, a near-field communication standard, a cellular standard, or any other wireless protocol via the respective communication interface devices 526, 546 and 564. The wireless communication links 587, 588 and 589 may be supplemented by or replaced with wired communication links 577, 578 and 579, respectively.

In some examples, the wearable drug delivery device 502 and/or the management device 506 may include a user interface 527 and 568, respectively, such as a keypad, a touchscreen display, levers, buttons, a microphone, a speaker, a display, or the like, that is operable to allow a user to enter information and allow the management device to output information for presentation to the user.

In various examples, the drug delivery system 500 may be an insulin drug delivery system. For example, the wearable drug delivery device 502 may be the OmniPod® (Insulet Corporation, Billerica, Mass.) insulin delivery device as described in U.S. Pat. Nos. 7,303,549, 7,137,964, or U.S. Pat. No. 6,740,059, each of which is incorporated herein by reference in its entirety.

In the examples, the drug delivery system 500 may implement the artificial pancreas (AP) algorithm (and/or provide AP functionality) to govern or control automated delivery of insulin to a user (e.g., to maintain euglycemia—a normal level of glucose in the blood). The AP application may be implemented by the wearable drug delivery device 502 and/or the sensor 504. The AP application may be used to determine the times and dosages of insulin delivery. In various examples, the AP application may determine the times and dosages for delivery based on information known about the user, such as the user's sex, age, weight, or height, and/or on information gathered about a physical attribute or condition of the user (e.g., from the sensor 504). For example, the AP application may determine an appropriate delivery of insulin based on glucose level monitoring of the user through the sensor 504. The AP application may also allow the user to adjust insulin delivery. For example, the AP application may allow a user to select (e.g., via an input) commands for output to the wearable drug delivery device 502, such as a command to set a mode of the wearable drug delivery device, such as an activity mode, a hyperglycemia protect mode, a hypoglycemia protect mode, deliver an insulin bolus, or the like. In one or more examples, different functions of the AP application may be distributed among two or more of the management device 506, the wearable drug delivery device (pump) 502 or the sensor 504. In other examples, the different functions of the AP application may be performed by one device, such the management device 506, the wearable drug delivery device (pump) 502 or the sensor 504. In various examples, the drug delivery system 500 may include features of or may operate according to functionalities of a drug delivery system as described in U.S. patent application Ser. No. 15/359,187, filed Nov. 22, 2016 and Ser. No. 16/570,125, filed Sep. 13, 2019, which are both incorporated herein by reference in their entirety.

As described herein, the drug delivery system 500 or any component thereof, such as the wearable drug delivery device may be considered to provide AP functionality or to implement an AP application. Accordingly, references to the AP application (e.g., functionality, operations, or capabilities thereof) are made for convenience and may refer to and/or include operations and/or functionalities of the drug delivery system 500 or any constituent component thereof (e.g., the wearable drug delivery device 502 and/or the management device 506). The drug delivery system 500—for example, as an insulin delivery system implementing an AP application—may be considered to be a drug delivery system or an AP application-based delivery system that uses sensor inputs (e.g., data collected by the sensor 504).

In an example, the drug delivery device 502 includes a communication interface device 564, which as described above may be a receiver, a transmitter, or a transceiver that operates according to one or more radio-frequency protocols, such as Bluetooth, Wi-Fi, a near-field communication standard, a cellular standard, that may enable the respective device to communicate with the cloud-based services 511. For example, outputs from the sensor 504 or the wearable drug delivery device (pump) 502 may be transmitted to the cloud-based services 511 for storage or processing via the transceivers of communication interface device 564. Similarly, wearable drug delivery device 502, management device 506 and sensor 504 may be operable to communicate with the cloud-based services 511 via the wireless communication link 588.

In an example, the respective receiver or transceiver of each respective device 502, 506 or mobile device may be operable to receive signals containing respective blood glucose measurement values of the number of blood glucose measurement values that may be transmitted by the sensor 504. The respective processor of each respective device 502, 506 or mobile device may be operable to store each of the respective blood glucose measurement values in a respective memory, such as 523, 563 or 573. The respective blood glucose measurement values may be stored as data related to the artificial pancreas algorithm, such as 529, 549, or 569. In a further example, the AP application operating on the management device 506 or sensor 504 may be operable to transmit, via a transceiver implemented by a respective communication interface device, 564, 574, 546, a control signal for receipt by a wearable drug delivery device. In the example, the control signal may indicate an amount of insulin to be expelled by the wearable drug delivery device 502.

In an example, one or more of the devices 502, 504, or 506 may be operable to communicate via a wired communication links 577, 578 and 579, respectively, with the cloud-based services 511 may utilize servers and data storage (not shown) to provide image recognition services as discussed above or the like. A communication link 599 that couples the drug delivery system 500 to the cloud-based services 511 may be a cellular link, a Wi-Fi link, a Bluetooth link, or a combination thereof, that is established between the respective devices 502, 504, or 506 of system 500. The cloud-based services may also be operable to provide processing services for the system 500, such as performing a process described with reference to one of the examples described with reference to FIGS. 2-4.

The wearable drug delivery device 502 may also include a user interface 527. The user interface 527 may include any mechanism for the user to input data to the drug delivery device 502, such as, for example, a button, a knob, a switch, a touch-screen display, or any other user interaction component. The user interface 527 may include any mechanism for the drug delivery device 502 to relay data to the user and may include, for example, a display, a touch-screen display, or any means for providing a visual, audible, or tactile (e.g., vibrational) output (e.g., as an alert). The user interface 527 may also include a number of additional components not specifically shown in FIG. 5 for sake brevity and explanation. For example, the user interface 527 may include a one or more user input or output components for receiving inputs from or providing outputs to a user or a caregiver (e.g., a parent or nurse), a display that outputs a visible alert, a speaker that outputs an audible, or a vibration device that outputs tactile indicators to alert a user or a caregiver of a potential activity mode, a power supply (e.g., a battery), and the like. Inputs to the user interface 527 may, for example, be a via a fingerprint sensor, a tactile input sensor, a button, a touch screen display, a switch, or the like. In yet another alternative, the activity mode of operation may be requested through a management device 506 that is communicatively coupled to a controller 251 of the wearable drug delivery device 502. In general, a user may generate instructions that may be stored as user preferences in a memory, such as 523 or 563 that specify when the system 500 is to enter the activity mode of operation.

Various operational scenarios and examples of processes performed by the system 500 are described herein. For example, the system 500 may be operable to implement process examples related to an activity mode including a hyperglycemia protect mode and a hypoglycemia protect mode as described in more detail below.

In an example, the drug delivery device 502 may operate as an artificial pancreas (AP) system (e.g., as a portion of system 500) and/or may implement techniques or an algorithm via an AP application that controls and provides functionality related to substantially all aspects of an AP system or at least portions thereof. Similarly, the management device 506 or mobile device 516 may also operate as an AP system with inputs from the early exercise detection application (such as 566 and 517, respectively). Accordingly, references herein to an AP system or AP algorithm may refer to techniques or algorithms implemented by an AP application executing on the drug delivery device 502, management device 506 or mobile device 516 to provide the features and functionality of an AP system. The drug delivery device 502, management device 506 or mobile device 516 may operate in an open-loop or closed-loop manner for providing a user with insulin.

In addition, the mobile device 516, as described in more detail in FIG. 1 as smartphone 100, may include a global positioning system that enables the determination of the location of the mobile device 516, which may be assumed to be collocated with the wearable drug delivery device 502. Alternatively, or in addition, the wearable drug delivery device 502 may also obtain location information by utilizing Wi-Fi location services obtained via communication interface device 526 enabling the controller 521 to determine the location of the wearable drug delivery device 502.

In an example, the early exercise detection algorithm may generate a history of indicators of early exercise detection, such as metadata information in image data that corresponds or correlates to exercise locations or times, or objects (which includes text) recognized from image data collected by the mobile device that indicates objects typically associated with exercise, such as exercise equipment (e.g., yoga balls, gym signs, weight machines, treadmills, stationary bikes, or the like), ballfields, track, or the like.

In an operational example, the processor 518 of the mobile device 516, when executing the early exercise detection application 517, may be operable to obtain image data from a camera (not shown in this example). The image data may include metadata obtained by the camera during an unlock procedure of the mobile device. The metadata may include location information or timestamp information. The processor 518 executing the early exercise detection application 517 may determine whether the obtained image data includes location information or timestamp information. Based on a determination that the metadata includes location information or a timestamp, the early exercise detection application may cause the processor 518 to evaluate the location information for a correspondence to known exercise locations or evaluate the timestamp with respect to another application, such as a calendar application 514, for a corresponding scheduled exercise time (e.g., a fitness class schedule downloaded to the calendar from a fitness center website or the like). The early exercise detection application may cause the processor 518 to evaluate the timestamp information for a correspondence to an exercise location based on a determination that the metadata includes location information or evaluate the timestamp information for a correspondence to an exercise diary based on a determination that the metadata includes timestamp information. The early exercise detection application may be further operable to determine whether any exercise related objects are recognized in the image data. The processor may identify a correspondence with an exercise location in the known exercise locations, an exercise time in the exercise diary or any exercise-related object recognized in the image data. In response to an identification of a correspondence, the processor may output an indication of early exercise detection to the artificial pancreas application. The artificial pancreas application executing on the mobile device 516 may operate in synchronization with the artificial pancreas application 569 executing on the management device 506 or with the artificial pancreas application 529 executing on the drug delivery device 502.

As discussed above, the artificial pancreas application may be operable to implement a diabetes treatment plan that involves the control the delivery of amounts or doses of insulin to the user. The diabetes treatment plan may include a number of parameters related to the delivery of insulin that may be determined and modified by a computer application referred to as an AP application. One of the number of parameters may be an indication of early exercise detection.

Returning to the operational example, the artificial pancreas application may receive the indication of early exercise detection from the early exercise detection application and, in response to receipt of the indication of early exercise detection, may calculate an insulin delivery adjustment amount. The artificial pancreas application executed by the processor may be operable to send a signal via a wireless communication link 586, delivering the calculated insulin delivery adjustment amount to the drug delivery device 502.

The controller 521 may be operable to execute instructions and may be operable to receive the signal from the mobile device indicating an amount of insulin to be delivered determined by the artificial pancreas application. The controller 521 may be operable to control the pump mechanism 524 via drive control signals. Based on the received signal from the mobile device indicating an amount of insulin to be delivered, the controller 521 may output a drive control signal to the pump mechanism to deliver the amount of insulin to be delivered.

In a more detailed example, the drug delivery device 502 may include a first electrode 571 and a second electrode 572 coupled to the controller 521. The first electrode 571 and the second electrode 572 may extend through a surface of the drug delivery device 502 and contact the skin of a user (not shown). The controller 521 may be operable to process signals received from the first electrode 571 and the second electrode 572 or may be operable to forward the received signal to the early exercise detection application 517 executing on the mobile device 516. In an operational example, the first electrode 571 and the second electrode 572 of a pair of electrodes may be positioned a predetermined distance apart (e.g., approximately 3-5 millimeters). The predetermined distance apart may be a distance substantially equal to a distance on the bottom and from substantially opposite ends of the wearable drug delivery device 502. The controller 521 (which is also a processor) may be operable to determine a value of a first electrical property (e.g., a voltage, a current, a resistance, a capacitance, or the like) between the pair of electrodes (571 and 572) coupled to a user. After a period of time has elapsed, the controller 521 may be operable to determine a value of a second electrical property between the pair of electrodes (571 and 572). The controller 521 may be operable to determine a difference between the value of the first determined electrical property and the value of the second determined electrical property. The difference in the values may be due, for example, to perspiration of the user or another condition that effects the respective electrical property. The controller 521 may be operable to determine that the difference corresponds to values of previously determined differences associated with a user exercising that have been stored in a user history database. For example, the values of previously determined differences may correspond to periods of known exercise by the user. In response determining the correspondence between the previously determined differences and periods of known exercise by the user, the controller 521 may be operable to generate and output a signal confirming the indication of early exercise detection.

The electrodes 571 and 572 were shown as being housed in the drug delivery device 502. However, in addition to the drug delivery device 502, or as an alternative, electrodes 551 and 552 may be housed in the blood glucose sensor 504, which may be a continuous blood glucose monitor, and may be operable to contact the skin surface of a user to enable detection of the electrical property, such as voltage, current, resistance or capacitance.

Various examples of an AP system include a wearable drug delivery device that may operate in the system to manage treatment of a diabetic user according to a diabetes treatment plan.

The techniques described herein for providing an early exercise detection application and response to an indication of early exercise detection as described herein for a drug delivery system (e.g., the smartphone 100 or system 500 or any components thereof) may be implemented in hardware, software, or any combination thereof. Any component as described herein may be implemented in hardware, software, or any combination thereof. For example, the smartphone 100 or system 500 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed devices may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as determining a purpose of a meal and providing instructions related an insulin dosage that is an appropriate response to the determined purpose of the meal. The dosage amount of insulin appropriate for compensating for the determined purpose of the meal may be reported to a user via a graphical user interface or the like communicatively coupled to the AP application or algorithm. Other open-loop actions may also be implemented by adjusting user settings or the like in an AP application or algorithm.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A non-transitory computer readable medium embodied with programming code executable by a processor, and the processor when executing the programming code is operable to perform functions, including functions to:
obtain image data including metadata from a camera coupled to the processor;
determine whether the obtained image data includes location information or timestamp information in the metadata;
based on a determination that the metadata includes location information, evaluate the location information for a correspondence to known exercise locations;
based on a determination that the metadata includes timestamp information, evaluate the timestamp information for a correspondence to an exercise diary;
identify a correspondence with either an exercise location in the known exercise locations or an exercise time in the exercise diary; and
in response to an identification of a correspondence, output an indication of early exercise detection to an artificial pancreas application.

2. The non-transitory computer readable medium of claim 1, further embodied with programming code executable by the processor, and the processor when executing the programming code is operable to perform further functions to:
in response to a correspondence not being identified, monitor for an unlock event to collect additional image data from a camera.

3. The non-transitory computer readable medium of claim 1, further embodied with programming code executable by the processor, and the processor when executing the programming code to obtain the image data, is operable to perform further functions, including functions to:
obtain first image data from a first camera, wherein the first camera is a forward facing camera;
obtain second image data from a second camera, wherein the second camera is a rear facing camera;
submit the obtained first image data and the obtained second image data to an object recognition process; and
receive an indication from the object recognition process that exercise-related objects are present in either the obtained first image data or the obtained second image data.

4. The non-transitory computer readable medium of claim 1, further embodied with programming code executable by the processor, and the processor when executing the programming code is operable to identify a correspondence with an exercise time reservation by performing functions to:
access an event manager application; and
identify events and exercise that a user has scheduled participation.

5. The non-transitory computer readable medium of claim 4, further embodied with programming code executable by the processor, and the processor when executing the programming code is operable to identify a correspondence with an exercise location in the known exercise locations by performing functions to:
obtain, via an input from a user interface, a name of the exercise location and a confirmation of a global positioning system indication of the exercise location; and
store the obtained name of the exercise location in a table of known exercise locations.

6. The non-transitory computer readable medium of claim 1, further embodied with programming code executable by the processor, and the processor when executing the programming code is operable to perform further functions to:
receive an input indicating a location of a mobile device;
obtain location information related to exercise;
compare the received input indicating the location of the mobile device; and
based on a result of the comparing, alter an insulin delivery adjustment amount.

7. The non-transitory computer readable medium of claim 1, wherein the processor is operable, when the programming code is executed by the processor, to perform further functions, including functions to:
determine a value of a first electrical property between a pair of electrodes coupled to a user, wherein a first electrode and a second electrode of the pair of electrodes are positioned a predetermined distance apart;
after a period of time has elapsed, determine a value of a second electrical property between the pair of electrodes;
determine a difference between the value of the first electrical property and the value of the second electrical property, wherein the difference is due to perspiration of the user;
determine that the difference corresponds to values of previously determined differences stored in a user history database, wherein the values of previously determined differences correspond to periods of known exercise by the user; and
output a signal confirming the indication of early exercise detection.

8. The non-transitory computer readable medium of claim 1, further embodied with programming code executable by the processor, and the processor when executing the programming code is operable to perform further functions to:
receive a blood glucose measurement value measured by a blood glucose monitor;
determine whether the received blood glucose measurement value falls within a predetermined threshold of an expected blood glucose measurement value, wherein the expected blood glucose measurement value was determined according to a first predictive blood glucose model; and
in response to the received blood glucose measurement value falling within the predetermined threshold, generate an early exercise indication based on a model determination.

9. The non-transitory computer readable medium of claim 8, wherein, when the programming code is executed by the processor, the processor is operable to perform further functions, including functions to:
in response to the early exercise indication based on the model determination, obtain another expected blood glucose measurement value determined according to a second predictive blood glucose model; and
determine whether the received blood glucose measurement value falls within a predetermined threshold of the other expected blood glucose measurement value; and
in response to the received blood glucose measurement value falling below the predetermined threshold of the other expected blood glucose measurement value, generate a confirmation of the early exercise indication; and
determine an insulin delivery adjustment amount based on the confirmation of the early exercise indication.

10. The non-transitory computer readable medium of claim 1, further embodied with programming code executable by the processor, and the processor when executing the programming code is operable to perform further functions to:
in response to the early exercise indication, determine a confidence level of a user's expected participation in exercise;
based on the determined confidence level, determine an insulin delivery adjustment amount for a next delivery of insulin; and
output instructions to deliver the determined insulin delivery adjustment amount.

11. The non-transitory computer readable medium of claim 10, wherein, when the programming code is executed by the processor, the processor is operable to perform further functions, including functions to:
receive signals from one or more movement-related sensors coupled to the processor;
determine whether any signals received from the movement-related sensors indicates exercise;
in response to a determination of an indication of exercise, increase the confidence level of the user's expected participation in exercise;
modify the insulin delivery adjustment amount based on the increase in the confidence level of the user's expected participation; and
output instructions to deliver a modified insulin delivery adjustment amount instead of the determined insulin delivery adjustment amount.

12. A system, comprising:
a mobile device including a processor, a transceiver, a camera, a memory, and programming code, an early exercise detection application and an artificial pancreas application stored in the memory, wherein the programming code, the early exercise detection application and the artificial pancreas application stored in the memory are executable by the processor, and when executing the early exercise detection application, the processor is operable to:
obtain image data from the camera, wherein the image data including metadata obtained by the camera during an unlock procedure of the mobile device, and the metadata including location information or timestamp information;
determine whether the obtained image data includes location information or timestamp information;
based on a determination that the metadata includes location information or a timestamp, evaluate the location information for a correspondence to an exercise location or evaluate the timestamp information for a correspondence to an exercise diary;
determine whether any exercise-related objects are recognized in the image data;
identify a correspondence of the location information with an exercise location in the known exercise locations, the timestamp information with an exercise time in the exercise diary or an exercise-related object recognized in the image data with exercise-related objects in the known exercise locations; and
in response to an identification of a correspondence, output an indication of early exercise detection to the artificial pancreas application; and
a wearable drug delivery device operable to deliver insulin to a user, including:
a communication interface device operable to receive and transmit signals;
a reservoir operable to store insulin;
a pump mechanism coupled to the reservoir and operable to expel the stored insulin from the reservoir in response to control signals;
a memory operable to store instructions; and
a controller operable to execute the instructions and control the communication interface device and the pump mechanism by outputting control signals and be communicatively coupled via the communication interface device to the transceiver and the processor of the mobile device, wherein the controller, when executing the instructions, is operable to:
receive a signal from the mobile device processor indicating an insulin delivery adjustment amount of insulin to be delivered as determined by the artificial pancreas application; and
output a drive control signal to the pump mechanism to deliver the insulin delivery adjustment amount of insulin.

13. The system of claim 12, wherein the processor when executing the early exercise detection application is operable to perform further functions to:
monitor for an unlock event to collect image data from a camera.

14. The system of claim 12, wherein the processor when executing the early exercise detection application is further operable to:
obtain first image data from a first camera, wherein the first camera is a forward facing camera;
obtain second image data from a second camera, wherein the second camera is a rear facing camera;
submit the obtained first image data and the obtained second image data to an object recognition process; and
receive an indication from the object recognition process that exercise-related objects are present in either the obtained first image data or the obtained second image data.

15. The system of claim 12, wherein the mobile device further comprises:
a global positioning system receiver and a Wi-Fi transceiver, wherein the processor is operable to determine a location of the mobile device based on signals received from the global positioning system receiver or the Wi-Fi transceiver.

16. The system of claim 15, wherein the processor when executing the early exercise detection application is operable to by performing functions to:
access a table of known exercise locations;
determine a correspondence between the image data and the location of known exercise locations;
based on a percentage of correspondence, generate a confidence level indicating a probability of a detection of exercise; and
utilize the confidence level in the determination of the insulin delivery adjustment amount of insulin.

17. The system of claim 12, wherein the wearable drug delivery device, further comprises:
a pair of electrodes coupled to a user, wherein a first electrode and a second electrode of the pair of electrodes are positioned a predetermined distance apart; and
wherein the controller is operable to:
detect a first electrical property between the pair of electrodes;
after a period of time has elapsed, detect a second electrical property between the pair of electrodes;
determine a difference between the first detected electrical property and the second detected electrical property, wherein the difference is due to perspiration of the user;
determine that the difference corresponds to values of previously determined differences stored in a user history database, wherein the values of previously determined differences correspond to periods of known exercise by the user; and
output a confirmation signal confirming that the user is exercising.

18. The system of claim 12, further comprising:
a blood glucose monitor communicatively coupled to the mobile device and operable to measure blood glucose of a user and output a blood glucose measurement value based on the measured blood glucose, wherein the processor of the mobile device is operable to:
receive the blood glucose measurement value from the blood glucose monitor;
determine whether the received blood glucose measurement value falls within a predetermined threshold of an expected blood glucose measurement value, wherein the expected blood glucose measurement value was determined according to a first predictive blood glucose model; and
in response to the received blood glucose measurement value falling within the predetermined threshold, generate an early exercise indication based on a model determination.

19. The system of claim 18, wherein the processor is operable to perform further functions, including functions to:
in response to the early exercise indication, obtain another expected blood glucose measurement value determined according to a second predictive blood glucose model; and
determine whether the received blood glucose measurement value falls within a predetermined threshold of the other expected blood glucose measurement value; and
in response to the received blood glucose measurement value falling below the predetermined threshold of the other expected blood glucose measurement value, generate a confirmation of the early exercise indication based on a model determination; and
determine an insulin delivery adjustment amount based on the confirmation of the early exercise indication.

20. The system of claim 12, wherein the mobile device further comprises:
one or more movement-related sensors coupled to the processor, and the processor is operable to perform further functions, including functions to:
receive signals from the one or more movement-related sensors;
determine whether any signals received from the one or more movement-related sensors indicates exercise;
in response to a determination of an indication of exercise, increase a confidence level of the user's expected participation in exercise;
modify the insulin delivery adjustment amount based on the increase in the confidence level of the user's expected participation; and
output instructions to the wearable drug delivery device to deliver the modified insulin delivery adjustment amount instead of the determined insulin delivery adjustment amount of insulin.
